SEQUENCE LISTING (12 sequences)

SEQ ID NO: 1 - protein, 12 aa, Oryctolagus cuniculus
EHKPGSPEVTGN

SEQ ID NO: 2 - protein, 9 aa, Oryctolagus cuniculus
KLHHGIQRH

SEQ ID NO: 3 - protein, 9 aa, Oryctolagus cuniculus
AALMNGSTL

SEQ ID NO: 4 - protein, 16 aa, Oryctolagus cuniculus
VEQSEHKPGSPEVTGN

SEQ ID NO: 5 - protein, 15 aa, Oryctolagus cuniculus
SRLMRSKLHHGIQRH

SEQ ID NO: 6 - protein, 19 aa, Oryctolagus cuniculus
AALMNGSTLLCGGSLLDTH

SEQ ID NO: 7 - protein, 10 aa, Oryctolagus cuniculus
LMTQDCVEQS

SEQ ID NO: 8 - protein, 10 aa, Oryctolagus cuniculus
MFCAGYLDGS

SEQ ID NO: 9 - protein, 10 aa, Oryctolagus cuniculus
TEWLSRLMRS

SEQ ID NO: 10 - protein, 3 aa, Oryctolagus cuniculus
PFP

SEQ ID NO: 11 - protein, 10 aa, Oryctolagus cuniculus
VCPKGECPWQ

SEQ ID NO: 12 - protein, 10 aa, Oryctolagus cuniculus
LCGGSLLDTH

(19) United States
(12) Patent Application Publication
(10) Pub. No.: US 2011/0250702 A1
(43) Pub. Date: Oct. 13, 2011

(54) ISOLATED PEPTIDES OF RABBIT FACTOR VII

(75) Inventors: Julien Gras, Les Ulis (FR); Annick Sauger, Les Ulis (FR)

(73) Assignee: LFB BIOTECHNOLOGIES, Les Ulis (FR)

(21) Appl. No.: 13/139,400

(22) PCT Filed: Dec. 31, 2009
(86) PCT No.: PCT/IB09/56003

§ 371 Date: Jun. 13, 2011

(30) Foreign Application Priority Data
Dec. 31, 2008 (FR) 08/07517

(51) Int. Cl. G01N 33/53; C07K 7/08; C07K 14/00; C07K 14/745; C07K 14/765; C07K 14/435; C07K 16/00; C07K 7/06; C07K 14/77

(52) U.S. Cl. 436/501; 530/328; 530/327; 530/326; 530/350; 530/363; 530/400; 530/387.9; 530/384

(57) ABSTRACT

The present disclosure relates to peptides isolated from rabbit factor VII and to the use of thereof for the generation of antibodies specifically directed against the latter. The disclosure also relates to the use of antibodies directed against rabbit factor VII for the detection or purification of rabbit factor VII, specifically when said rabbit factor VII is in a biological sample which also contains human factor VII.

### CROSS-REFERENCE TO RELATED APPLICATIONS

This application is a National Phase Entry of International Application No. PCT/IB2009/056003, filed on Dec. 31, 2009, which claims priority to French Patent Application Serial No. 08/07517, filed on Dec. 31, 2008, both of which are incorporated by reference herein.

### FIELD OF THE INVENTION

The present invention relates to peptides isolated from the rabbit factor VII and to their uses for generating antibodies specifically directed against the latter. The invention also relates to the use of antibodies directed against the rabbit factor VII for detecting or purifying the rabbit factor VII in particular when said rabbit factor VII is present in a biological sample jointly containing human factor VII.

### BACKGROUND

The production of recombinant proteins in a transgenic animal henceforth is an alternative for producing widely used proteins. When the transgenic recombinant protein is intended to be administered to patients, the purity and the innocuousness of the administered transgenic recombinant protein preparation are particularly important.

Many methods for purifying or detecting a recombinant protein are based on the affinity and specificity of a compound capable of binding to said recombinant protein. These steps for purifying or detecting a recombinant protein are most particularly delicate when said recombinant protein may be present, concomitantly with a strongly homologous protein. Indeed, when the recombinant protein is found in solution with one or several proteins homologous to this recombinant protein, it then becomes difficult to develop detection or purification tools with which a high level of discrimination may be achieved between the recombinant protein of interest and the undesirable homologous protein(s), by using standard purification or detection techniques.

The difficulty of purifying or specifically detecting proteins having homology between them is encountered when a transgenic recombinant protein is produced in a transgenic organism or micro-organism which naturally also expresses a homologous protein of said transgenic protein. Notably, recombinant proteins are targeted, produced in organisms naturally having in their genome, a so-called “orthologous” gene coding for a strongly homologous protein to the recombinant protein coded by a transgene.

It is common that a human or animal transgenic protein expressed in a transgenic animal is the homolog of an endogenous protein naturally expressed in the transgenic animal. The production of the homologous endogenous natural protein represents a major technical drawback in situations where it is sought to prevent co-extraction or co-detection of the transgenic protein and of the homologous endogenous natural protein. When the transgenic recombinant protein consists in a protein of therapeutic interest, intended for making a drug, the presence, in the purified preparation of the recombinant protein, of any homologous endogenous protein of the transgenic protein may cause undesirable effects for the patient to whom the drug is administered, including the mediation of an undesirable immune response against the contaminating natural protein, which may reduce the efficiency of the medical treatment and even sometimes cause autoimmune responses which may endanger the life of the patient. Such problems are more and more often encountered with increasing resort to the production of therapeutic transgenic proteins in transgenic animals.

In order to propose therapeutic products having high innocuousness, transgenic proteins therefore have to be specifically purified in the presence of very small amounts of undesirable homologous proteins, and if possible in total absence of undesirable homologous proteins. It is therefore necessary to have tools which allow detection or purification of an endogenous protein of a transgenic animal, homologous to the exogenous the protein of interest, both proteins being likely to be contained in a same biological sample of the transgenic animal.

U.S. Pat. No. 5,861,491 proposes a method for separating human lactoferrin from cow milk containing bovine lactoferrin. This method is based on chromatography of hydrophobic interactions. This chromatography uses a resin which contains a butyl group or a phenyl group which is used as a ligand itself bound to an agarose support.

European patent EP 1 181 351 proposes a method for separating heterologous proteins present in the milk of a transgenic animal (human HSA and bovine BSA). The method is based on the suppression of the expression of the endogenous protein of the transgenic animal by replacement of the gene coding for the endogenous protein with a DNA sequence coding for the heterologous polypeptide. This molecular biology method is particularly cumbersome to apply.

The present invention proposes antibodies specifically directed against the rabbit factor VII for detecting and/or purifying said rabbit factor VII, which may be again found in a biological sample of a transgenic rabbit, intended for producing human factor VII.

### SUMMARY

The object of the present invention is peptides isolated from the rabbit factor VII, the amino acid sequence of which is selected from EHKPGSPEVTGN (SEQ ID NO:1), KLHHGIQRH (SEQ ID NO:2) and AALMNGSTL (SEQ ID NO:3). These peptides respectively correspond to the sequences of amino acids from amino acid 354 to amino acid 365, from amino acid 433 to amino acid 441 and from amino acid 207 to amino acid 215 of the protein sequence of the rabbit factor VII (Oryctolagus cuniculus) accessible under the access number P98139 in the Swissprot database. These three peptides are all comprised between the amino acids 1192 to P444 corresponding to the heavy chain of the rabbit factor VII.

The object of the present invention is also a polypeptide formed by a peptide according to the invention, and by at least one additional oligopeptide comprising from 1 to10 amino acids placed at either one or both N-terminal and C-terminal ends of said peptide. Within the scope of the present invention, the term of polypeptide therefore refers to a sequence of amino acids including from 10 to 32 amino acids, preferably 15 to 32 amino acids, and comprising one of the peptides of the invention. The size of the polypeptide of the invention is selected so as to optimize the immunogenicity of the peptides of the invention.

In a preferred embodiment of the present invention, the polypeptide of the invention comprises at least one additional oligopeptide, the amino acids of which are selected in the N-terminal or C-terminal flanking region of the peptide of the invention, when reference is made to the protein sequence of the rabbit factor VII accessible under the access number P98139. The polypeptide is therefore formed by “lengthening” the peptide of the invention by selecting additional amino acids naturally contiguous to the N-terminal and/or C-terminal end of this peptide in the sequence of the rabbit factor VII. When the peptide of the invention has the sequence EHKPGSPEVTGN (SEQ ID NO:1), the amino acids forming the oligopeptide added to the N-terminal end of the peptide are therefore selected from the sequence LMTQDCVEQS (SEQ ID NO:7) and/or the amino acids forming the oligopeptide added to the C-terminal end are therefore selected from the sequence MFCAGYLDGS (SEQ ID NO:8). When the peptide of the invention has the sequence KLHHGIQRH (SEQ ID NO:2), the amino acids forming the oligopeptide added to the N-terminal end of the peptide are therefore selected from the sequence TEWLSRLMRS (SEQ ID NO:9) and/or the amino acids forming the oligopeptide added to the C-terminal end are therefore selected from the sequence PFP (SEQ ID NO:10). Finally, when the peptide of the invention has the sequence AALMNGSTL (SEQ ID NO:3), the amino acids forming the oligopeptide added to the N-terminal end of the peptide are therefore selected from the sequence VCPKGECPWQ (SEQ ID NO: 11) and/or the amino acids forming the oligopeptide added to the C-terminal end are therefore selected from the sequence LCGGSLLDTH (SEQ ID NO:12).

More preferably, the polypeptide of the invention has the sequence:

VEQSEHKPGSPEVTGN (SEQ ID NO:4), i.e. the N-terminal end of the peptide of sequence EHKPGSPEVTGN (SEQ ID NO:1) is lengthened with 4 amino acids (naturally contiguous to this peptide in the protein sequence of the rabbit factor VII),

SRLMRSKLHHGIQRH (SEQ ID NO:5), i.e. the N-terminal end of the peptide of sequence KLHHGIQRH (SEQ ID NO:2) is lengthened with 6 amino acids (naturally contiguous to this peptide in the protein sequence of the rabbit factor VII), or

AALMNGSTLLCGGSLLDTH (SEQ ID NO:6), i.e. the C-terminal end of the peptide of sequence AALMNGSTL (SEQ ID NO:3) is lengthened with 10 amino acids (naturally contiguous to this peptide in the protein sequence of the rabbit factor VII).

Another object of the present invention is a chimeric protein, comprising at least one peptide of the invention and/or at least the one polypeptide of the invention, in combination with a vector protein, said peptide(s) and/or said polypeptide(s) and said vector protein being optionally separated by a spacer. In a preferred embodiment, the chimeric protein of the invention comprises, as a vector protein, a protein selected from keyhole limpet hemocyanin (KLH), bovine serum albumin (BSA), ovalbumin (OVA) and bovine thyroglobulin (THY). In a preferred embodiment, a cystein may be added to the N-terminal end of the peptides or polypeptides in order to promote the combination or grafting of the vector protein via the use of thiol chemistry well-known to one skilled in the art.

In a preferred embodiment, the chimeric protein of the invention comprises peptides of SEQ ID NOS:1, 2 and 3 or polypeptides of SEQ ID NOS: 4, 5 and 6, said peptides or said polypeptides being sequentially organized and separated from each other by a spacer. The spacer allowing separation of the peptides and polypeptides from each other is selected according to techniques well-known to one skilled in the art. In a preferred embodiment, the chimeric protein of the invention comprises as a vector protein, a protein selected from keyhole limpet hemocyanin (KLH), bovine serum albumin (BSA), ovalbumin (OVA) and bovine thyroglobulin (THY). The vector protein may equally occupy the N- or C-terminal end of the chimeric protein of the invention.

The object of the present invention is also a peptide composition for inducing production of antibodies comprising at least one peptide of SEQ ID NOS:1, 2 and/or 3, and/or at least one polypeptide of the invention and/or at least one chimeric protein of the invention. When the peptide composition according to the invention comprises several peptides and/or polypeptides of the invention, the latter may appear in an individualized form or in a concatenated form. When the composition according to the invention comprises several peptides and/or polypeptides of the invention in a concatenated form, the peptides and/or polypeptides may be separated from each other by a spacer.

The peptide composition according to the invention may also comprise an adjuvant. The use of an adjuvant may be required for increasing the intensity or the duration of the immune response and thereby allow reduction in the amount of the peptide/polypeptide/chimeric protein per dose or in the total number of doses required for ensuring immunity. The adjuvants which may be used within the scope of the invention are in a non-limiting way, aluminium salts (hydroxide, phosphate, sulfate, calcium salts or bacterial products). The peptide composition of the invention gives the possibility of inducing in a host animal, antibodies specifically directed against the rabbit factor VII.

The peptides, polypeptides or chimeric proteins according to the invention may also be used for carrying out screening of antibodies specifically recognizing the rabbit factor VII. The object of the present invention is also an antibody or an antibody functional fragment directed against at least one of the peptides, at least one of the polypeptides, or at least one of the chimeric proteins described above and capable of specifically recognizing the rabbit factor VII (and in particular the species, Oryctolagus cuniculus). Such an antibody may be polyclonal or monoclonal. By antibody functional fragment is meant a Fab fragment, a Fab′ fragment, a F(ab)2 fragment or an scFv fragment (Blazar et al., 1997, Journal of Immunology 159: 5821- 5833 et Bird et al., 1988, Science 242: 423-426).

By specific recognition, is meant in the sense of the present invention that the antibodies directed against at least one of the peptides, at least one of the polypeptides or at least one of the chimeric proteins of the invention are capable of binding to the rabbit factor VII but are not capable of binding to the factor VII of another species. Preferably, the antibodies of the invention may bind to the rabbit factor VII but cannot bind to the human factor VII, and therefore allow discrimination of the rabbit factor VII from the human factor VII.

In order to produce polyclonal antibodies, the peptides, polypeptides or chimeric proteins according to the invention are synthesized and injected into a host animal, which may be a mouse, a rabbit or any other animal known to one skilled in the art for producing antibodies. After this so-called immunization step, the sera of the host animal are harvested and purified so as to obtain polyclonal antibodies.

The purification of the antibodies from the sera may be accomplished by affinity chromatography, by precipitation with aluminium sulfate, by ion exchange chromatography, by filtration on a gel or by any other technique known to one skilled in the art. In a particular embodiment of the invention, said polyclonal antibodies are separated from the other constituents of the serum by affinity chromatography on a column, on which is bound a peptide, a polypeptide or a chimeric protein according to the invention, which will be recognized by the generated antibodies.

In order to produce monoclonal antibodies, the peptides, polypeptides or chimeric proteins according to the invention are synthesized and injected into a host animal, which may be a mouse, a rabbit or any other animal known to one skilled in the art for producing antibodies. After this so-called immunization step, the animals expressing antibodies directed against the peptides of the invention are once again immunized before the intended date of the fusion step intended for preparing a hybridoma. The spleen of the animals is sampled in order to recover the B lymphocytes which produce the antibodies directed against the peptides of the invention and the B lymphocytes are fused with cancer cells in order to form hybridomas. The best clones are then sub-cloned in order to allow production of the monoclonal antibody of the invention. The antibody or the antibody functional fragment of the invention may be used for purposes of detection or purification of the rabbit factor VII, in particular when said rabbit factor VII is contained in a biological sample also containing the human factor VII.

Another object of the invention also relates to a method for detecting and/or quantifying the rabbit factor VII which may be present in a biological sample containing human factor VII and which may contain rabbit factor VII, preferably a biological sample taken from a transgenic rabbit, said transgenic rabbit being intended for producing human factor VII, characterized in that it comprises the steps of:

putting said biological sample in contact with an antibody or an antibody functional fragment according to the invention under conditions allowing the formation of a complex between the rabbit factor VII and said antibody or said antibody functional fragment and not allowing the formation of a complex between the human factor VII and said antibody or said antibody functional fragment; and

detecting and/or quantifying the formation of said complex by any suitable means.

The detection of the rabbit factor VII may be carried out with any suitable means known to one skilled in the art and, in particular, with a sandwich ELISA technique or a surface plasmon resonance technique (Biacore). A detection method according to the invention may further conventionally comprise the steps:

of immobilizing a monoclonal anti-rabbit factor VII antibody of the invention, for example on a chip;

of depositing the sample to be tested; and

of revelation by means of a polyclonal biotinylated anti-factor VII antibody or by measuring the binding level of the sample to be tested with the monoclonal antibody of the invention.

Another object of the invention also relates to a method for purifying the human factor VII from a biological sample containing the human factor VII and which may contain the rabbit factor VII, preferably a biological sample taken from a transgenic rabbit, characterized in that it comprises the steps of:

putting said biological sample in contact with the antibody or the antibody functional fragment according to the invention under conditions allowing the formation of a complex between the rabbit factor VII and said antibody or said antibody functional fragment and not allowing the formation of a complex between the human factor VII and said antibody or said antibody functional fragment,

separating the human factor VII from said complex formed by the rabbit factor VII and said antibody or said antibody functional fragment.

The purification method according to the invention may further conventionally comprise the steps of:

immobilizing the monoclonal anti-rabbit factor VII antibody of the invention (or a functional fragment of the latter) on an affinity support (gel or magnetic bead):

putting the biological sample containing the human factor VII and which may contain the rabbit factor VII in the presence of the immobilized monoclonal antibody; and

separating the human factor VII from said complex formed by the rabbit factor VII and said antibody or said antibody functional fragment after a suitable contacting time.

In a preferred embodiment of the invention, the biological sample stems from a transgenic female rabbit, said transgenic female rabbit being intended for producing human factor VII. The biological sample therefore contains the human factor VII, described as a protein of interest, and may contain a protein homologous to the human factor VII and notably rabbit factor VII. Advantageously, the biological sample is a body fluid, a cell, a cell homogenate, a tissue, a tissue homogenate, an organ or an entire organism. Preferably, the biological sample is a liquid biological sample such as blood, a derivative of blood (blood derivative), milk or milk derivative. This may be plasma, plasma cryoprecipitate, clarified milk or derivatives thereof. Advantageously, the transgenic rabbit produces the human transgenic factor VII in its mammary glands under the control of a specific promoter allowing expression of said transgenic protein in the milk of said transgenic rabbit.

An example of a method for preparing protein in the milk of a mammalian female other than a human being is given in document EP 0 527 063, the teaching of which may be taken for producing the protein of the invention. A plasmid containing the WAP (Whey Acidic Protein) promoter is made by introducing a sequence including the promoter of the WAP gene, this plasmid being made so as to be able to receive a foreign gene placed under the dependency of the WAP promoter. The plasmid containing the promoter and the gene coding for the protein of the invention are used for obtaining transgenic female rabbits, by micro-injection into the male pronucleus of female rabbit embryos. The embryos are then transferred into the oviduct of hormonally prepared females. The presence of the transgenes is revealed by the Southern technique from the DNA extracted from the obtained transgenic young rabbits. The concentrations in the milk of the animals are evaluated by means of specific radio-immunological tests.

Other documents describe methods for preparing proteins in the milk of a mammalian female other than humans. Mention may be made, without any limitation, of the documents U.S. Pat. No. 7,045,676 (transgenic mouse) and EP 1 739 170 (Von Willebrand factor production in a transgenic mammal), notably mentioning the promoter of casein.

### BRIEF DESCRIPTION OF THE DRAWINGS

FIG. 1: Tracking of the immunization of a mouse. Representative curves of the proportion of antibodies directed against the rabbit FVII produced by one of the immunized mice with the peptides/polypeptides of the invention. The serum samplings are carried out on the day of the humanization (J0), and then 21, 35 and 85 days from the immunization (J21, J35 and J85). The presence of antibodies directed against rabbit FVII in the serum of immunized mice is tested by an ELISA test of the “direct” type, wherein the wells of the plate are coated with recombinant rabbit FVII (Am. Diagnostica). The absorbance of each well is measured and the measured values are plotted in ordinates on the graph of FIG. 1. For each taken serum sample, the absorbance is measured for different dilutions.

FIG. 2: Immunological detection of FVII by Western blot under non-reduced conditions (no cutting of the disulfide bridges between both chains of the FVII). A rabbit plasma FVII (well A), a recombinant rabbit FVII (well B) and a human transgenic FVII (well C) are separated on a polyacrylamide gel of the 4-12% SDS-PAGE type, concomitantly with a molecular weight marker (noted as “Std PM” for molecular weight standard). The gel is transferred onto a membrane, and then a Western blot is performed by using the immunoserum of an immunized mouse with the peptides/polypeptides of the invention, or with a polyclonal sheep antibody directed against human FVII capable of also recognizing rabbit FVII.

FIG. 3: Immunological detection of FVII by a Western blot under reduced conditions (cutting of the disulfide bridges between both chains of the FVII). A rabbit plasma FVII (well A), a recombinant rabbit FVII (well B) and a human transgenic FVII (well C) are separated on polyacrylamide gel of the 4-12% SDS-PAGE type, concomitantly with a molecular weight marker (noted as “Std PM” for molecular weight standard). The gel is transferred on a membrane, and then a Western blot is performed by using the immunoserum of a mouse immunized with the peptides/polypeptides of the invention, or with a polyclonal sheep antibody directed against the human FVII also capable of recognizing the rabbit FVII.

### EXAMPLES

### Example 1

### Selection of Immunogenic Peptides for Preparing Antibodies Specifically Recognizing Rabbit Factor FVII

Immunogenic peptides intended for preparing antibodies specifically directed against the rabbit factor FVII are selected from the protein sequence of rabbit factor FVII bearing the access number P98139 in the Swiss Prot protein base. Three peptides are retained:

the peptide of sequence EHKPGSPEVTGN (SEQ ID NO: 1);

the peptide of sequence KLHHGIQRH (SEQ ID NO:2); and

the peptide of sequence AALMNGSTL (SEQ ID NO:3).

The accessibility to the solvent of these peptides is analyzed so as to make sure that none of them is buried within the tertiary structure of the protein. Moreover, a complementary analysis shows that none of these peptides seem to include any glycosylation sites.

a—Preparation of the Polypeptides used for Applying Immunization:

The selected peptides are “lengthened” by selecting additional amino acids naturally present at the N-terminal and/or C-terminal end of each peptide in the rabbit factor FVII sequence in order to obtain a polypeptide having a size of at least 15 amino acids, thereby allowing optimization of the immunogenicity of the peptides of the invention during immunization. The N-terminal end of the peptide of sequence EHKPGSPEVTGN (SEQ ID NO:1); is lengthened with 4 amino acids (naturally contiguous to this peptide in the protein sequence of the rabbit factor VII) in order to obtain the polypeptide of sequence VEQSEHKPGSPEVTGN (SEQ ID NO:4).

The N-terminal end of the peptide of sequence KLHHGIQRH (SEQ ID NO:2) is lengthened with 6 amino acids (naturally contiguous to this peptide in the protein sequence of the rabbit factor VII) in order to obtain the polypeptide of sequence SRLMRSKLHHGIQRH (SEQ ID NO:5). Finally, the C-terminal end of the peptide of sequence AALMNGSTL (SEQ ID NO:3) is lengthened with 10 amino acids (naturally contiguous to this peptide in the protein sequence of the rabbit factor VII) in order to obtain the polypeptide of sequence AALMNGSTLLCGGSLLDTH (SEQ ID NO:6).

b—Construction of Chimeric Proteins used for Immunizing the Mice:

The peptides VEQSEHKPGSPEVTGN (SEQ ID NO:4), SRLMRSKLHHGIQRH (SEQ ID NO:5) and AALMNGSTLLCGGSLLDTH (SEQ ID NO:6) are synthesized and chemically coupled to KLH (Keyhole Limpet Hemocyanin), a vector protein (Calbiochem).

### Example 2

### Immunization of Mice

Each of the chimeric proteins formed from the peptides of the invention is then used for immunizing a batch of 6 mice with 3 injections at days J0, J15 and J30, J45. The immunizations are performed with one of the coupled peptides or by mixing two of the coupled peptides or the three peptides coupled together. The mice sera are tested as to their capability of binding the corresponding peptide(s) of the invention or the polypeptide(s) which were coupled to the vector protein in order to form the chimeric protein used for immunization. The tests used for evaluating the obtained sera consist in techniques of the ELISA or Biacore type and are described in Example 3.

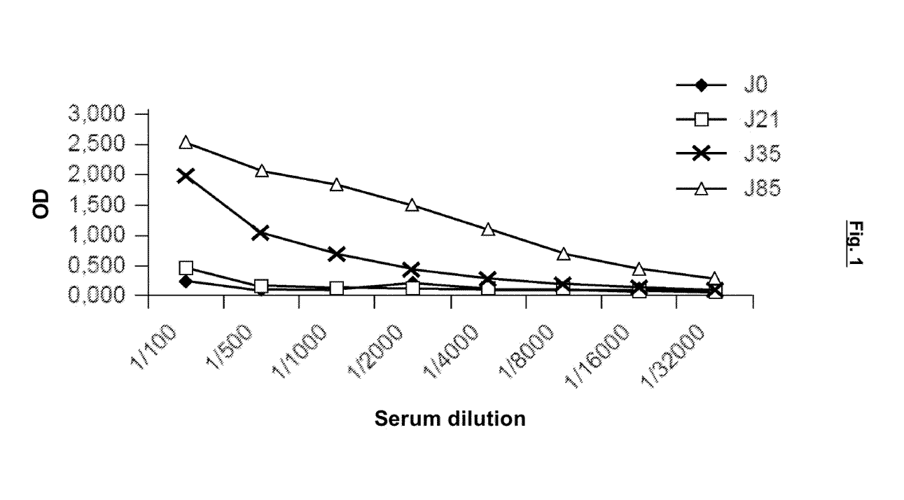

FIG. 1 shows the development of immunization by means of serum samplings at days J21, J35 and J85. The presence of antibodies directed against rabbit FVII in the serum of immunized mice is tested with an ELISA test of the “direct” type, wherein the wells of the plate are covered (“coated”) with recombinant rabbit FVII (Am. Diagnostica). The absorbance of each well is measured and the measured values are plotted in ordinates on the graph of FIG. 1. The increase in the absorbance expresses an increase in the antibodies directed against recombinant rabbit FVII in the serum of the mice.

a—Preparation of Polyclonal Antibodies Specifically Directed Against the Rabbit Factor FVII

In order to obtain the polyclonal antibodies directed against either one of the peptides of the invention, the mice which are positively tested are sacrificed and the totality of the serum of these mice is purified by affinity chromatography by using a column on which are grafted the peptide(s) (or polypeptide(s)) of the invention corresponding to the obtained serum. The polyclonal antibodies bound on the column are then eluted by modifying their binding affinity for the peptides grafted on the column, according to techniques well-known to one skilled in the art.

b—Preparation of Monoclonal Antibodies Specifically Directed Against the Rabbit Factor FVII

The selected mouse or mice expressing antibodies directed against the peptides of the invention are immunized once again, 3 days before the date intended for the fusion step for preparing a hybridoma. The spleen of the mice is sampled in order to recover the B lymphocytes which produce the antibodies directed against the peptides of the invention. The B lymphocytes are fused with myelomatous cells of the SP20 line in order to form hybridomas.

After fusion with SP20, the cells are distributed in cell culture plates in a selected medium. Screening of the fusion wells is carried out by measuring the respective binding of rabbit FVII and of human FVII to the antibodies produced by the fusions distributed in the different wells.

Table 1 shows the absorbance values reflecting the binding of the antibodies stemming from the fusion wells, with rabbit FVII and with human FVII. The results displayed in Table 1 clearly demonstrate that the antibodies obtained via immunization by means of chimeric proteins comprising the peptides of the invention have affinity which is significantly greater for rabbit FVII.

TABLE 1

| Name | (Recombinant) rabbit FVII OD | Human FVII OD |
|---|---|---|
| 1C8 | 1.383 | 0.415 |
| 1F10 | 1.322 | 0.432 |
| 1F11 | 1.085 | 0.295 |
| 1F12 | 1.566 | 0.375 |
| 2B6 | 0.934 | 0.298 |
| 2B8 | 1.054 | 0.283 |
| 2B10 | 0.853 | 0.240 |
| 2D8 | 0.994 | 0.199 |
| 3A4 | 1.898 | 0.166 |
| 3C10 | 1.062 | 0.264 |
| 3D10 | 0.941 | 0.142 |
| 5B4 | 0.87 | 0.191 |
| 5B5 | 1.406 | 0.292 |
| 5B6 | 0.924 | 0.277 |
| 5C6 | 1.372 | 0.233 |
| 5D5 | 1.244 | 0.368 |
| 5D9 | 0.813 | 0.151 |
| 5E6 | 1.248 | 0.158 |
| 5E7 | 0.912 | 0.144 |
| 5E11 | 0.878 | 0.144 |
| 7B8 | 1.099 | 0.068 |
| 8B2 | 0.925 | 0.215 |
| 8F8 | 1.029 | 0.174 |
| 8G12 | 1.074 | 0.154 |

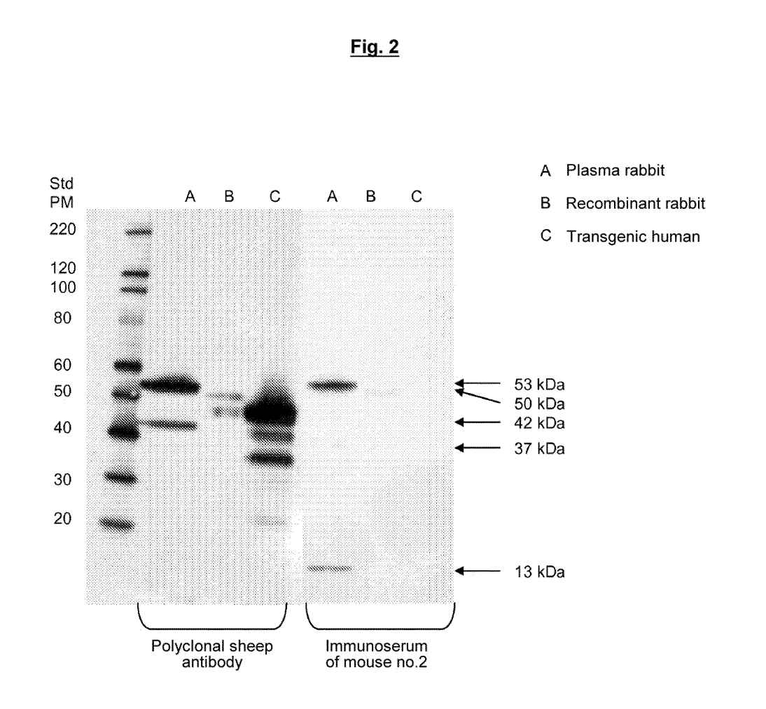

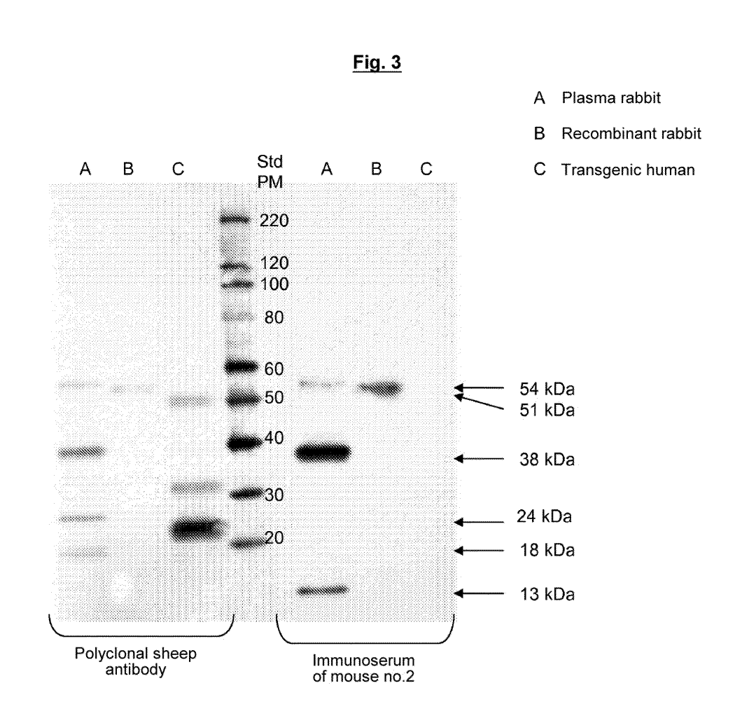

These results are confirmed by immunological detection tests (Western blots) performed with the immunoserum of a mouse immunized with the peptides/polypeptides of the invention. The results of these tests are copied into FIGS. 2 and 3. A plasma rabbit FVII (well A), a recombinant rabbit FVII (well B) and a human transgenic FVII (well C) are separated on a polyacrylamide gel of the 4-12% SDS-PAGE type, concomitantly with a molecular weight marker (noted as “Std PM” for molecular weight standard), under non-reduced conditions (see FIG. 2) or under reduced conditions (see FIG. 3). The gel is transferred onto a membrane, and then a Western blot is carried out by using the immunoserum of a mouse immunized with the peptides/polypeptides of the invention, or with a polyclonal sheep antibody directed against human FVII also capable of recognizing rabbit FVII.

The polyclonal sheep antibody directed against human FVII and capable of recognizing rabbit FVII allows detection of rabbit plasma FVII, recombinant rabbit FVII and human transgenic FVII at a time, under reduced and non-reduced conditions. A contrario, it clearly appears that the antibodies present in the immunoserum of the mouse as for them only allow detection of rabbit plasma FVII and rabbit recombinant FVII but do not allow detection of human FVII. It should be noted that the rabbit recombinant FVII is produced in the form of a single chain, which is therefore not dissociated upon separation on a gel under reduced conditions. The rabbit plasma FVII and the human transgenic FVII are as for them dissociated into light and heavy chains during the separation under reduced conditions.

The results of FIGS. 2 and 3 therefore confirm that the antibodies prepared from the peptides/polypeptides of the invention allow specific recognition of rabbit FVII, and they do not recognize human FVII. The fusions having the best results (which have a greater affinity for the rabbit factor VII are cloned by limiting dilution and new ELISA differential screening is carried out. The best clones are then sub-cloned. The cells allowing production of the monoclonal antibodies of the invention are then put back into culture and the lysate or the cell supernatant containing the monoclonal antibodies according to the invention are prepared according to techniques well-known to one skilled in the art.

### Example 3

### Screening of the Obtained Antibodies

The screening of the produced (either polyclonal or monoclonal) antibodies is carried out by applying two successive tests based on an ELISA technique of the “sandwich” type. The first test consists of detecting antibodies which recognize the rabbit factor VII and comprises the steps of:

immobilizing a polyclonal antibody recognizing both the rabbit factor VII and the human factor VII in a culture plate.

depositing a rabbit plasma sample in order to bind the rabbit factor VII onto the polyclonal antibody.

depositing a sample containing the monoclonal or polyclonal antibody to be tested obtained after immunization of a host animal (see Example 2).

revealing the binding of said antibody with a biotinylated anti-host animal (here the mouse) antibody.

The second test consists of detecting the antibodies which recognize the human factor VII and comprises the steps of:

immobilizing a polyclonal antibody recognizing both the rabbit factor VII and the human factor VII in a culture plate.

depositing a human plasma sample in order to bind the human factor VII onto the polyclonal antibody.

depositing a sample containing the monoclonal or polyclonal antibody to be tested obtained after immunization of a host animal (see Example 2).

revealing the binding of said antibody with a biotinylated anti-host animal (here the mouse) antibody.

The ELISA “sandwich” procedure is carried out by using a microplate in polyvinyl chloride (PVC). 50 μL of a solution (at 2 μg/mL in PBS) of a polyclonal antibody recognizing both the rabbit factor VII and the human factor VII are added in each well. About 100 ng of this antibody will bind to the PVC in each well (i.e. about 300 ng of antibodies/cm²). The microplate is left at 4° C. overnight in order to allow the binding of a maximum of antibodies.

The wells are then washed twice with PBS. The remaining sites are saturated with a saturation buffer consisting of PBS and of 3% BSA (Bovine Serum Albumin), for a duration of at least two hours under a humid atmosphere at room temperature. The wells are then washed twice with PBS. 50 μL of the antigen solution (here, the rabbit plasma sample in the first test or the human plasma sample in the second test) are added into the wells and incubated for at least 2 hours under a humid atmosphere and at room temperature. The plates are washed four times with PBS.

A fraction of the sample containing the monoclonal or polyclonal antibody to be tested (obtained after immunization of the mice) is then added. Several dilutions of this sample are tested, ranging from preferably 1:10 to 1:10,000 and comprising conventionally the dilutions 1:10, 1:100, 1:1,000 and 1:10,000. The selection of the suitable dilutions for proceeding with the screening of the antibodies of the invention may be based on preliminary binding tests which are well-known to one skilled in the art.

The sample containing the monoclonal or polyclonal antibody to be tested is incubated in contact with the microplates for at least 2 hours under a humid atmosphere at room temperature. The wells are then washed several times with PBS. The biotinylated anti-mouse antibody is then added according to the recommendations of the manufacturer, at a dilution of 1:1,000, in a PBS buffer containing 0.5% of Tween 20. When the recommended incubation time is reached with the biotinylated anti-mouse antibody, it is then proceeded with the detection of the amount of monoclonal or polyclonal antibody to be tested which has been bound.

The ELISA “sandwich” type techniques are well-known to one skilled in the art and may easily be modified for adjusting the amounts and concentrations of each of the antibodies and/or antigen for attaining the desired result. An antibody according to the invention will be characterized in that it has a positive response to the first test (it detects the rabbit factor VII) and a negative response to the second test (it does not detect the human factor VII).

As an alternative to the “sandwich” ELISA test, a “direct” ELISA test may also be used in order to achieve screening of the produced (either polyclonal or monoclonal) antibodies. This test is characterized by the fact that the rabbit FVII or the human FVII are directly deposited in the wells of the plates in order to cover (coat) the surface. The FVII used may be obtained either by plasma fractionation or by genetic recombination.

The “direct” ELISA procedure is carried out by using a microplate in polyvinyl chloride (PVC). 100 μL of a solution (at 1 μg/mL in PBS) of rabbit factor VII or human factor VII are added into each well, which corresponds to about 100 ng of FVII (i.e. about 300 ng of FVII/cm²). The microplate is left at 4° C. overnight in order to allow the binding of a maximum of FVII.

The wells are then washed twice with PBS. The remaining sites are saturated with a saturation buffer consisting of PBS and of 3% BSA (Bovine Serum Albumin), for a duration of at least 2 hours under a humid atmosphere at room temperature. The wells are then washed twice with PBS.

A fraction of the sample containing the monoclonal or polyclonal antibody to be tested (obtained after immunization of the mice) is then added. Several dilutions of this sample are tested, preferably ranging from 1:10 to 1:10,000, and conventionally comprising the dilutions 1:10, 1:100, 1:1,000 and 1:10,000. The selection of the suitable dilutions for proceeding with screening of the antibodies of the invention may be based on preliminary binding tests which are well-known to one skilled in the art.

The sample containing the monoclonal or polyclonal antibody to be tested is incubated in contact with the microplates for at least 2 hours under a humid atmosphere at room temperature. The wells are then washed several times with PBS. The biotinylated anti-mouse antibody is then added according to recommendations of the manufacturer, at a dilution of 1:1,000, in a PBS buffer containing 0.5% of Tween 20. When the recommended incubation time is reached with the biotinylated anti-mouse antibody, it is then proceeded with the detection of the amount of monoclonal or polyclonal antibody to be tested which has been bound.

The “direct” ELISA type techniques are well-known to one skilled in the art and may be easily modified in order to adjust the amounts and concentrations of each of the antibodies and/or antigen in order to attain the desired results. An antibody according to the invention will be characterized in that it has a much greater response to a test in which the wells are coated with rabbit FVII as compared with a test in which a human FVII is immobilized in the wells.

As an alternative to ELISA tests, any molecular interaction test may be used for the screening. The molecular interaction tests which may be used are notably characterized by two possible configurations. In a first configuration, a rabbit FVII is immobilized onto which the antibodies to be screened are injected. A human FVII is used as a reference. The relative signal of the interaction of the tested antibodies with the rabbit FVII as compared with the interaction with the human FVII is recorded. The second configuration comprises the immobilization of the antibodies to be screened, notably via the use of a protein A or an anti-mouse antibody, followed by sequential injection of rabbit and human FVII. Each injection is separated by a so-called regeneration step during which the formed antibody-FVII interactions are dissociated. The molecular interaction tests may notably be carried out on surface plasmon resonance systems (Biacore) or of the Quartz Microbalance type.

### Example 4

### Detection of the Rabbit Factor VII from Transgenic Rabbit Milk used for Producing the Human Factor VII

The monoclonal or polyclonal antibodies of the invention which have a specificity for rabbit FVII (no binding to human FVII) in the tests of Example 3 therefore allow specific detection of rabbit factor VII including when the latter is found in a biological sample which may also contain the human factor VII. These polyclonal or monoclonal antibodies may in particular be applied in a method for detecting, and if necessary quantifying the rabbit factor VII in the milk of transgenic female rabbits used for producing the human factor VII. The techniques for detecting a protein in a sample, based on polyclonal or monoclonal antibodies directed against this protein are well-known to one skilled in the art, who may without any difficulty adapt them to the use of the monoclonal or polyclonal antibodies of the invention.

In a preferred embodiment the sample containing the monoclonal or polyclonal antibodies of the invention is deposited in a microplate in polyvinyl chloride (PVC) at a dilution of 1:100. The microplate is left at 4° C. overnight. The wells are then washed twice with PBS, and then the free remaining sites are saturated with a saturation buffer consisting of PBS and 3% BSA (Bovine Serum Albumin) for a duration of at least two hours under a humid atmosphere at room temperature. The wells are then washed twice with PBS.

50 μL of rabbit milk from a transgenic female rabbit used for producing human factor VII are added into the wells and incubated for at least 2 hours under a humid atmosphere and at room temperature. Preferentially, different dilutions of the transgenic female rabbit milk are deposited in the wells of the micro plate. The dilutions generally used are the following: 1, 1:10, 1:50, 1:100, 1:500, and 1:1,000, however, other dilutions may easily be used. The transgenic female rabbit milk may also be the subject of one or several pre-purification steps before being used for detecting the rabbit factor VII therein. The plates are then washed four times with PBS.

A solution of monoclonal or polyclonal antibodies directed against the rabbit factor VII and coupled with peroxidase is added into the wells and is incubated in contact with the microplates for at least 2 hours under a humid atmosphere at room temperature. The dilution of antibodies coupled with peroxidase in the solution used is generally 1:1,000 in a PBS buffer containing 0.5% of Tween 20. The wells are then washed several times with PBS. The antibody coupled with the peroxidase used may be a commercially available antibody (one will then act according to the recommendations of the manufacturer) or an antibody according to the invention which will have been coupled beforehand with peroxidase.

Detection is carried out by adding a solution containing orthophenylenediamine (OPD-H₂O₂). The solution containing OPD is incubated with the microplates at room temperature for about 3 minutes. In the presence of peroxidase, the addition of the OPD solution causes the occurrence of a coloration revealing the presence of rabbit factor VII in the tested transgenic milk. The reaction is stopped by means of a stopping reagent (3M H₂SO₄ or 1M HCl) and the optical density of the reaction mixture is read within a period of time from 10 minutes to 2 hours after stopping the reaction by means of a microplate spectrophotometric reader. The absorbance at 492 nm is measured (the blank being adjusted on the contents of a well not having been incubated in the presence of transgenic milk). The intensity of the coloration is proportional to the amount of antibody coupled with peroxidase and therefore to the amount of rabbit factor VII bound on the solid phase.

Preferentially, in parallel with the preparation of the microplate intended to be put into contact with the transgenic female rabbit milk, it is also proceeded with the preparation of a microplate put into contact with a range of increasing concentrations of rabbit factor VII. The procedure remains similar to the one described above. Upon detection, the microplate put into contact with the rabbit factor VII concentration range allows a calibration curve to be plotted, corresponding to the development of absorbance versus the factor VII concentration. The rabbit factor VII concentration in the transgenic female rabbit milk is determined by plotting the absorbance value measured on the calibration curve.

### Example 5

### Extraction and Purification of the Factor VII Contained in the Milk of Transgenic Female Rabbits Producing human Factor VII

Non-skimmed raw milk of transgenic female rabbit producing human factor VII is diluted with 0.25 M sodium phosphate buffer, pH 8.2 and centrifuged at 10,000 g for 1 hour at 15° C. After centrifugation, three phases are present: a surface lipid phase (cream), a clear non-lipid aqueous phase enriched with factor VII (majority phase) and a solid white sedimented phase (precipitates of insoluble caseins and calcium compounds). The non-lipid aqueous phase containing the factor VII is collected and then filtered on a sequence of filters having a pore size from 1 μm to 0.45 μm.

The filtered non-lipid aqueous phase is then dialyzed on an ultrafiltration membrane in order to make it compatible with the chromatography phase. The non-lipid aqueous phase containing the factor VII is then purified by chromatography on a hydroxyapatite gel, followed by 100 kDa tangential filtration and a 50 kDa concentration/dialysis. During the tangential filtration, the factor VII passes through the membrane having a porosity of 100 kDa, while the proteins of high molecular weight (i.e. with a molecular weight of more than 100 kDa) are as for them retained. With this treatment it is possible to notably reduce the risks of proteolytic hydrolysis during the subsequent purification steps. The resulting solution containing the factor VII is then purified via 3 successive chromatographies on an ion exchanger gel Q-Sepharose Fast Flow (QSFF) carried out for purifying and concentrating the factor VII and allowing activation of the factor VII into an activated factor VII (factor VIIa).

During the first chromatography, carried out on Q-Sepharose FF gel, the factor VII-rich protein fraction is eluted with a buffer comprising 0.05M calcium chloride at pH 7.5 (“high calcium” elution) and also allows activation of factor VII into a factor Vila. After dialysis, the eluate is then separated on a Q-Sepharose FF 2 column. During this step, a fraction containing the very high purity factor VII is eluted with a buffer containing 0.005 M calcium chloride at pH 7.5 (“low calcium” elution). With this step it is possible to remove more than 95% of the accompanying proteins (proteins from the female rabbit milk).

Finally, the solution containing the factor VII is separated on Q-Sepharose FF 3. During this step, the factor VII is then eluted with a buffer containing sodium chloride 0.28 M at pH 7.0 (“sodium” elution). The composition of the factor VII which results from this elution has a degree of purity of more than 95%. The product is then compatible with injection via an intravenous route.

### Example 6

### Purification of a Solution Containing the Rabbit Factor VII and the Human Factor VII on a Resin Grafted with the Antibodies of the Invention Specifically Directed Against the Rabbit Factor VII

The polyclonal or monoclonal antibodies of the invention are grafted on a column so that they may be used for achieving an affinity chromatography. Preferably, the column used is a column of the “protein A” type or of the “protein G” type. The grafting is carried out according to the recommendations of the manufacturer and, as this is well-known to one skilled in the art, the applied procedure may be adapted to the selected type of column. The primary amines which may present in the sample containing the monoclonal or polyclonal antibodies of the invention are removed by filtration on resin or by dialysis against a phosphate buffer. The agarose-protein G resin is re-suspended and then washed and equilibrated with the washing buffer containing 50 mM of sodium borate, pH 8.2. The antibody solution (at about 100 μg/mL) is placed in contact with the resin and the mixture is gently homogenized. The resin/antibody mixture is then cast into a column. The column is then washed twice with washing buffer. Disuccinimidyl suberate (DSS) is suspended in DMSO or DMF and one equivalent volume of 0.1 M phosphate buffer, containing 0.15 M of NaCl, pH 7.2 is added. This mixture is deposited on the column, and delicately homogenized with the resin for 1 hour at room temperature. The column is then washed with phosphate buffer 0.1 M, containing 0.15 M of NaCl, pH 7.2. Blocking buffer comprising 0.1 M of ethanolamine is then added onto the column in order to block any still activated ester group. The resins/blocking resin mixture is slowly homogenized for 10 minutes at room temperature, and then buffer containing a primary amine (at pH 2.8) is passed over the column, in order to elute any antibody which is not covalently bound to the protein G. The column is then washed twice with washing buffer before its use for affinity chromatography.

In order to carry out the affinity chromatography, the column is equilibrated with PBS type buffer at pH 7.2. The solution containing the rabbit factor VII (and corresponding to any of the steps for purifying the factor VII mentioned above) is diluted (1v/1v) by adding PBS. This diluted solution is then passed over the column and the column is washed with PBS buffer until the absorbance at 280 nm returns to the base line.

The non-retained solution of the column, now specifically without any rabbit factor VII, is recovered. The absence of rabbit factor VII is checked for example by immunological reaction. This non-retained solution, which only contains the human factor VII produced by the transgenic female rabbits, may then be concentrated, purified and/or conditioned in order to prepare a human factor VII composition for therapeutic use.

1. An isolated peptide comprising having as an amino acid sequence one of the sequences is selected from one of: EHKPGSPEVTGN (SEQ ID NO:1), KLHHGIQRH (SEQ ID NO:2) and AALMNGSTL (SEQ ID NO:3).

2. An isolated polypeptide formed by a peptide according to claim 1, and by at least one additional oligopeptide of 1 to 10 amino acids, placed at the either one or both of the N-terminal and C-terminal ends of said peptide.

3. The isolated polypeptide according to claim 2, wherein, when the peptide has the sequence EHKPGSPEVTGN (SEQ ID NO:1), the amino acids making up the oligopeptide added to the N-terminal end are selected from the sequence LMTQDCVEQS (SEQ ID NO:7) and/or the amino acid is making up the oligopeptide added to the C-terminal end are selected from the sequence MFCAGYLDGS (SEQ ID NO:8).

4. The isolated polypeptide according to claim 2, wherein, when the peptide has the sequence KLHHGIQRH (SEQ ID NO:2), the amino acids making up the oligopeptide added to the N-terminal end are selected from the sequence TEWLSRLMRS (SEQ ID NO:9) and/or the amino acids making up the oligopeptide added to the C-terminal end are selected from the sequence PFP (SEQ ID NO:10).

5. The isolated polypeptide according to claim 2, wherein, when the peptide has the sequence AALMNGSTL (SEQ ID NO:3), the amino acids making up the oligopeptide added to the N-terminal end are selected from the sequence VCPKGECPWQ (SEQ ID NO: 11) and/or the amino acids making up the oligopeptide added to the C-terminal end are selected from the sequence LCGGSLLDTH (SEQ ID NO:12).

6. The polypeptide according to claim 2, further comprising a sequence of amino acids selected from one of: VEQSEHKPGSPEVTGN (SEQ ID NO:4), SRLMRSKLHHGIQRH (SEQ ID NO:5) and AALMNGSTLLCGGSLLDTH (SEQ ID NO:6).

7. A chimeric protein, comprising at least one peptide according to claim 1, in combination with a vector protein, the peptide(s) and the vector protein being optionally separated by a spacer.

8. The chimeric protein according to claim 7, wherein the vector protein is selected from one of: keyhole limpet hemocyanin (KLH), bovine serum albumin (BSA), ovalbumin (OVA) and bovine thyroglobulin (THY).

9. A peptide composition for introducing production of antibodies comprising at least one peptide according to claim 1 and/or at least one chimeric protein.

10. An antibody or antibody functional fragment specifically directed against at least one of the peptides according to claim 1, at least one of the polypeptides, or at least one of the chimeric proteins.

11. The antibody according to claim 10, wherein the antibody is polyclonal or monoclonal.

12. The antibody functional fragment according to claim 10, consisting in a Fab fragment, a Fab′ fragment, a F(ab)2 fragment or an scFv fragment.

13. The use of a peptide according to claim 1, of a polypeptide according to one of claims 2 to 6, or of a chimeric protein polypeptide or of a chimeric protein, both for the screening of antibodies directed against the rabbit factor VII.

14. The use of an antibody or of an antibody functional fragment according to claim 11, for detecting and purifying au rabbit factor VII.

15. The use according to claim 14, wherein the rabbit factor VII is contained in a biological sample also containing the human factor VII.

16. A method for detecting and/or quantifying rabbit factor VII, which may be present in a biological sample taken from a transgenic rabbit, said transgenic rabbits being intended for producing the human factor VII, the method comprising:
putting the biological sample in contact with an antibody or an antibody functional fragment under conditions allowing the formation of a complex between the rabbit factor VII and the antibody or the antibody functional fragment and not allowing the formation of a complex between the human factor VII and the antibody or the antibody functional fragment; and
detecting and/or quantifying the formation of the complex.

17. A method for purifying human factor VII, from a biological sample taken from a transgenic rabbit, the method comprising:
putting the biological sample in contact with an antibody or an antibody functional fragment under conditions allowing the formation of a complex between the rabbit factor VII and the antibody or the antibody functional fragment and not allowing the formation of a complex between the human factor VII and the antibody or the antibody functional fragment; and
separating the human factor VII from the complex formed by the rabbit factor VII and the antibody or the antibody functional fragment.
